(19) United States
(12) Patent Application Publication
(10) Pub. No.: US 2005/0028829 A1
(43) Pub. Date: Feb. 10, 2005

(54) Spectrum for dietary supplementation selection system and method following human olfactory

(76) Inventors: John Louis Kitkoski, Spokane, WA (US); Elsie Anita Kitkoski, Spokane, WA (US)

Correspondence Address: Elsie A. Kitkoski, 103 W. 39th Ave., Spokane, WA, 99203-1529, US

(21) Appl. No.: 10/794,867

(22) Filed: Mar. 8, 2004

Related U.S. Application Data
(60) Provisional application No. 60/492,448, filed on Aug. 5, 2003.

(51) Int. Cl. A61B 19/00

(52) U.S. Cl. 128/898

(57) ABSTRACT

The mammalian olfactory apparatus is able to recognize and distinguish thousands of structurally diverse volatile chemicals. This chemosensory function is mediated by a very large family of seven-transmembrane olfactory (odorant) receptors encoded by approximately 1,000 genes, the majority of which are believed to be pseudogenes in humans. THEREFORE, a system and method for use to aid a person in taking “dietary supplements” by following human olfactory receptor repertoire protocol. The methodology comprises of “smell sensitive dietary supplements” set out in a spectrum following human olfactory receptor repertoire protocol and human body biochemical absorption of said dietary supplement and olfactory reception. Said dietary supplements are arranged to provide specific olfactory information to aid the person to stop or modify any adverse health-related behavior. The basic component of the methodology consists of a self administered approach to correcting vitamin and mineral deficiencies or imbalances. Numerical indica smell table is used for each person to score each dietary supplement, with a score of one through five take one dietary supplement or a score of five through ten do not take said dietary supplement. Numerical indicia score is used to indicate how often a person is to go through the system: with a score of said dietary supplements of one's a person is to go through the entire system three times a day. Score dietary supplements a two but no scores of one, person goes through the entire system twice a day. Score dietary supplements three, person can go through the system once a day. With all scores of four's and five's a person can go through the system three time a week and with score's of six through ten the person goes through the system one time a week.

Said dietary supplements are formulated with minimal binders and no sugars, artificial colors, or preservatives The bottles of each dietary supplement is numbered 1 thru 20 in an order (spectrum) that relates to the way olfactory neurons stimulates and to the protocols of human body absorption. The supplements, in tablet and capsule form, are “smell sensitive”.

Each subject can determine which dietary supplement to take by how it smells. Those that smell good to mild should indicate the body has a need for that dietary supplement at that time. Those that smell bad indicate the body has an adequate or excess amount of that dietary supplement at that time.

SMELL TABLE: As subjects smell each dietary supplement, it is rated on a scale of 1 to 10. Take the dietary supplement that score 1-5, don't take the dietary supplement that score 6-10.
Score of (1) Very good—Smells very good, sweet. (2) Good—smells good, quite pleasant.
Score of (3) No smell—no smell at all. Score of (4) Okay, Mild—some smell, but mild.
Score of (5) So-so—Smell is not good, not bad. (6) Not so good.
(7) Bad—Smells bad. Score of (8) Real bad—definitely smells bad.
Score of (9) Awful—Smells worse than bad, it's awful. Score of (10) Puquey! Yuk!—could not smell worse.

### BACKGROUND OF THE INVENTION

The present invention relates to selection of dietary supplements in sealed, olfactory—isolating tablets/containers and in particular, to a system and method that indicate deficiencies or excesses and/or selection based on olfactory cues.

Bio-chemical pathways of the warm-blooded animals and humans absorb specific dietary supplements in an order that is specific: The olfactory odor receptors pick up odors in a specific order.

Chemical sensitivity which enables an organism to react to molecules in the external environment and facilitate intake and avoidance for self preservation and protection is present in one form or another in all animals.

Human chemosensory receptors of smell have a regulatory role with regard to nutrient intake. Nutrients important in human nutrition may be classified in three categories; water soluble elements, fat soluble elements, and water itself. Except for linoleic and linolenic aids, fats and cholesterol need not be considered in this scheme as they can be manufactured internally.

The sense of smell functions as the primary component for regulating the intake of fat soluble nutrients. To chemically stimulate the olfactory cell, an element must be volatile, slightly water soluble, and definitely fat soluble. Even though human olfactory receptors are some of the least sensitive in the animal kingdom, they can still identify a billionth of an ounce of chemical floating in the air. This high discriminating capacity, compared to the sense of taste which can recognize only four or five tastes, suggest that the sense of smell may function as an early detection system for fat soluble nutrients about to enter the systemic circulation. As on chews, tastes, and swallows food, molecules of nutrients can waft up the nasopharynx and stimulate the receptors of the olfactory nerve, sending signals to the brain with regards to the nature and concentration of different chemicals passing down the throat. The importance of this in regulation of nutrient intake becomes obvious when one considers the fact that most of what one perceives as the flavor of a food is actually secondary to the stimulation of olfactory receptors. The finding that human infants change sucking behavior and increased and decreased intake during breast feeding due to detection of garlic flavor and alcohol respectively in mother's milk supports the possibility of regulation of intake secondary to retronasal perception of volatile elements present in food.

### BRIEF SUMMARY OF THE INVENTION

An object of the present invention is to provide olfactory information corresponding to the dietary supplement need of the warm-blooded animals or humans.

Another object of the present invention is to provide a method and/or system to facilitate dietary supplement selection by warm-blooded animals or humans.

A preferred embodiment of the present invention employs a smell table and a smell order based on olfactory protocols.

The dietary supplements 1 thru 20 are based on the bio-chemical pathways and rate of absorption in a spectrum following the olfactory protocols.

Warm-blooded animals or humans can sniff the open containers and show a preference for a dietary supplement selection.

As used hereinafter, the term “Spectrum for Dietary Supplementation” refers to the 1 thru 20 dietary supplement kit of the present invention, said 1 thru 20 dietary supplement kit has a unique “Spectrum or Order” starting with #1 then #2 then #3 and so on. If the subject skip's around from #1 then to #5 then to #11 and then sniff's #3 the “Spectrum” or “Order” is broken, Olfaction is disrupted and absorption is compromised.

The olfactory helps to indicate deficiencies or excesses. The dietary supplements serve as facilitators of enzyme functions. The need is dependent upon the metabolism of the individual. Dietary supplements are not stored to any great extent, so they need to be replaced almost daily.

1. The human and animal body absorbs dietary supplements in a specific order.
The human and animal olfactory odor receptors pick up odors in a specific order.
Object of the invention: To put forth a system of deciphering the dynamics of smelling in humans and animals with the bio-chemical pathways of human and animal body absorption of specific dietary supplements.
A method of delivery of dietary supplementation following olfactory in warm blooded animals and humans to those concentrations found in a warm-blooded animal and human.
Use of the olfactory odor receptor to indicate which dietary supplement is needed by warm-blooded animal or human at that time based on smell. The olfactory sense helps to indicate deficiencies or excesses of dietary supplementation.
A method of delivery of dietary supplementation following olfactory in warm-blooded animals and humans without increasing the risk of over supplementation, comprising:
Warm-blooded animals and humans olfactory neurons have to have order. Warm-blooded animals and humans cannot smell vitamin A and then smell vitamin D and get the desired results. Remembering that the shape of the dietary supplement molecules determines how they smell. The order of the dietary supplement cannot be compromised do to the fact that some dietary supplements will cancel each other out of the smell table.
A methodology for delivery of dietary supplementation by using “smell sensitive” dietary supplements in an order that relates to the way the warm-blooded animal and human bodies absorb that specific dietary supplement which can determine which dietary supplement to take by how they smell.
